(19) United States
(12) Patent Application Publication
(10) Pub. No.: US 2023/0293628 A1
(43) Pub. Date: Sep. 21, 2023

(54) MEANS AND METHODS FOR TREATING BACTERIAL INFECTIONS

(71) Applicants: FORSCHUNGSZENTRUM BORSTEL LEIBNIZ-ZENTRUM FUR MEDIZIN UND BIOWISSENSCHAFTEN, Borstel (DK); FREIE UNIVERSITAT BERLIN, Berlin (DE); UNIVERSITAT ZU LUBECK, Lubeck (DE); RHEINISCH-WESTFALISCHE TECHNISCHE HOCHSCHULE (RWTH) AACHEN, Aachen (DE)

(72) Inventors: Klaus BRANDENBURG, Hamburg (DE); Lena HEINBOCKEL, Hamburg (DE); Norbert REILING, Ahrensburg (DK); Guillermo MARTINEZ DE TEJADA, Pamplona (ES); Tobias SCHURHOLZ, Aachen (DE); Karl MAUSS, Hamburg (DE); Gunther WEINDL, Berlin (DE)

(21) Appl. No.: 18/150,042

(22) Filed: Jan. 4, 2023

Related U.S. Application Data
(62) Division of application No. 15/999,531, filed on Aug. 17, 2018.

(30) Foreign Application Priority Data
Feb. 19, 2016 (EP) 16156556.9

(51) Int. Cl. A61K 38/16; A61P 17/02; A61P 31/04; A61P 31/06; A01N 63/50; A01N 37/18; A01N 43/16; A01N 43/90; A61K 31/43; A61K 31/431; A61K 31/496; A61K 31/546; A61K 31/65; A61K 31/7036; A61K 31/7048; A61K 38/10; A61K 38/14; C07K 7/08

(52) U.S. Cl. CPC A61K 38/16; A61P 17/02; A61P 31/04; A61P 31/06; A01N 63/50; A01N 37/18; A01N 43/16; A01N 43/90; A61K 31/43; A61K 31/431; A61K 31/496; A61K 31/546; A61K 31/65; A61K 31/7036; A61K 31/7048; A61K 38/10; A61K 38/14; C07K 7/08

(57) ABSTRACT

The present invention relates to a pharmaceutical composition comprising or consisting of a combination of (a) two or more peptides, each peptide consisting of or comprising 17 to 23 amino acids, wherein the amino acids in positions 1 to 23, counted from the N-terminus, are as follows (1) G, S or lacking; (2) C or lacking; (3) K or R; (4) K or R; (5) Y, W or F; (6) K or R; (7) K or R; (8) F, W or L; (9) K or R; (10) K or L or lacking; (11) W, L or F; (12) K or R; (13) F, Y or C; (14) K or R; (15) G or Q; (16) K or R; (17) F, L or W; (18) F or W; (19) F, L or W; (20) W or F; (21) C or lacking; (22) F or G or lacking; (23) G or lacking; or (b) one or more peptides, each peptide consisting of or comprising 17 to 23 amino acids, wherein the amino acids in positions 1 to 23, counted from the N-terminus, are as follows (1) G, S or lacking; (2) C or lacking; (3) K or R; (4) K or R; (5) Y, W or F; (6) K or R; (7) K or R; (8) F, W or L; (9) K or R; (10) K or L or lacking; (11) W, L or F; (12) K or R; (13) F, Y or C; (14) K or R; (15) G or Q; (16) K or R; (17) F, L or W; (18) F or W; (19) F, L or W; (20) W or F; (21) C or lacking; (22) F or G or lacking; (23) G or lacking, and one or more antibiotics selected from small organic molecule antibiotics such as ceftriaxone, oxacillin, amoxicillin, amikacin, ciprofloxacin, erythromycin, imipenem and tetracycline, and peptidic antibiotics such as daptomycin and vancomycin.

### CROSS-REFERENCE TO RELATED APPLICATIONS

This patent application is the Divisional Patent Application of U.S. patent application Ser. No. 15/999,531, filed Aug. 17, 2018, which is the National Phase of International Application No. PCT/EP2017/053487, filed Feb. 16, 2017, which designated the U.S., and that International Application was published under PCT Article 21(2) in English, which claims the benefit of priority to European Patent Application No. 16156556.9, filed Feb. 19, 2016. The entire contents of the foregoing applications are incorporated herein by reference, including all text, tables, sequence listings and drawings.

### SEQUENCE LISTING

The contents of the electronic sequence listing (009848-0573114_SEQUENCE_LISTING.xml; 3,622 bytes; and Date of Creation: Jan. 3, 2023) is herein incorporated by reference in its entirety.

The present invention relates to a pharmaceutical composition comprising or consisting of a combination of (a) two or more peptides, each peptide consisting of or comprising 17 to 23 amino acids, wherein the amino acids in positions 1 to 23, counted from the N-terminus, are as follows (1) G, S or lacking; (2) C or lacking; (3) K or R; (4) K or R; (5) Y, W or F; (6) K or R; (7) K or R; (8) F, W or L; (9) K or R; (10) K or L or lacking; (11) W, L or F; (12) K or R; (13) F, Y or C; (14) K or R; (15) G or Q; (16) K or R; (17) F, L or W; (18) F or W; (19) F, L or W; (20) W or F; (21) C or lacking; (22) F or G or lacking; (23) G or lacking; or (b) one or more peptides, each peptide consisting of or comprising 17 to 23 amino acids, wherein the amino acids in positions 1 to 23, counted from the N-terminus, are as follows (1) G, S or lacking; (2) C or lacking; (3) K or R; (4) K or R; (5) Y, W or F; (6) K or R; (7) K or R; (8) F, W or L; (9) K or R; (10) K or L or lacking; (11) W, L or F; (12) K or R; (13) F, Y or C; (14) K or R; (15) G or Q; (16) K or R; (17) F, L or W; (18) F or W; (19) F, L or W; (20) W or F; (21) C or lacking; (22) F or G or lacking; (23) G or lacking, and one or more antibiotics selected from small organic molecule antibiotics such as ceftriaxone, oxacillin, amoxicillin, amikacin, ciprofloxacin, erythromycin, imipenem and tetracycline, and peptidic antibiotics such as daptomycin and vancomycin.

In this specification, a number of documents including patent applications and manufacturer's manuals is cited. The disclosure of these documents, while not considered relevant for the patentability of this invention, is herewith incorporated by reference in its entirety. More specifically, all referenced documents are incorporated by reference to the same extent as if each individual document was specifically and individually indicated to be incorporated by reference.

Severe bacterial infections are an ever-increasing threat worldwide. This is aggravated by the continued appearance of multi-resistant bacteria on the one hand and a lack of suitable novel antibiotic agents on the other hand. Owing to an ever-increasing number of elderly patients, the number of bacterial infections which are difficult to treat increases at a yet faster pace. For example, in Germany there are about 150,000 infections with MRSA (methicillin-resistant Staphylococcus aureus), about 10% of which are lethal owing to bacterial sepsis (according to Deutsche Sepsis-Gesellschaft, Infection, 41, Suppl. 1, Weimar Sepsis Update 2013). Experts estimate that about 50,000 patients per year die in German hospitals owing to an infection with resistant bacteria. Globally, the annual number of lethal cases of sepsis is estimated to be between 9 and 10 million. Further infections such as Mycobacterium tuberculosis present a serious problem for public health systems owing to lengthy therapies and enormous costs. Yet about 1 million patients die per year from this infection. The continued appearance of resistant strains (such as MDR and XDR strains) further complicates the situation.

On the other hand, also non-systemic bacterial infections require increasingly lengthy treatments. Morbidity owing to such non-systemic bacterial infections keeps increasing. Examples include erysipelas, impetigo, folliculitis, boil, and carbuncle. The latter mentioned infections are usually not life-threatening, yet they significantly impact quality of life. Other non-systemic bacterial infections presenting a particular challenge for public health systems are those which cause chronic inflammation. Examples include chronic obstructive pulmonary disease (COPD). In particular, Haemophilus influenzae is known to be involved in chronic inflammation accompanying COPD.

EP 2 271 356 describes antimicrobial peptides. This document fails to suggest the excellent performance of peptides in the treatment of the specific medical indication which is the bacterial infection of badly healing wounds. Moreover, this document entirely fails to suggest a synergism with regard to antibacterial activity when combining two or more peptides or combining one or more peptides with known antibiotics, typically small organic molecule antibiotics and instead reports additive effects.

Heinbockel et al. (Antimicrobial agents and chemotherapy 57, 1480-1487 (2013)) describes an antimicrobial peptide with high endotoxine neutralization capacity. In the hands of the authors of Heinbockel et al., the mentioned peptidic antibiotic failed to exhibit synergy when used together with small organic molecule antibiotics, even at the highest concentrations used.

Antimicrobial peptides for the treatment of wound infections and skin diseases have been described in, for example, Duplantier and van Hoek, Frontier in Immunology 4, 1-14 (2013), Kenshi and Richard, Eur. J. Dermatol. 18, 11-21 (2008) and Mangoni et al. (doi: 10.1111/exd.12929). The described peptides are fundamentally different in terms of structure from the agents of the present invention.

The technical problem underlying the present invention can be seen in the provision of improved means and methods for the treatment of bacterial infections, in particular infections by multi-resistant bacteria, including wound infections, skin infections and sepsis.

This technical problem has been solved by the subject-matter of the claims enclosed herewith.

In a first aspect, the present invention relates to a peptide comprising or consisting of the sequence of SEQ ID NO:1 (GKKYRRFRWKFKGKLFLFG). The peptide consisting of this sequence is also referred to as peptide 19-4LF or Aspidasept II.

The term “peptide” generally describes linear molecular chains of amino acids containing up to 30 amino acids covalently linked by peptide bonds. The total number of amino acids comprised in the peptide may increase to preferably up to 30 if one or more amino acids are added to the sequence of SEQ ID NO: 1 at the N- and/or C-terminus. Said amino acids may or may not contribute to the functionality of the peptide. In other words, amino acid(s) added may or may not confer a distinct function to the peptide, be it its antimicrobial activity or another function.

The number of amino acids may further increase if the peptide of the invention is fused to another peptide or to a polypeptide. Fusion constructs are described in EP 2 271 356. Peptides may form oligomers consisting of at least two identical or different molecules. The corresponding higher order structures of such multimers are, correspondingly, termed homo- or heterodimers, homo- or heterotrimers etc.

The one-letter code abbreviations as used to identify amino acids throughout the present invention correspond to those commonly used for amino acids.

The peptides of the present invention can be produced synthetically.

Chemical synthesis of peptides is well known in the art. Solid phase synthesis is commonly used and various commercial synthesizers are available, for example automated synthesizers by Applied Biosystems Inc., Foster City, CA; Beckman; or MultiSyntech, Bochum, Germany. Solution phase synthetic methods may also be used. For example, peptide synthesis can be carried out using N-α-9-fluorenylethoxycarbonyl amino acids and a preloaded trityl resin or an aminomethylated polystyrene resin with a p-carboxytritylalcohol linker. Couplings can be performed in dimethylformamide using N-hydroxybenzotriazole and 2-(1H-benzotriazole-1-yl)-1,1,3,3-tetramethyluronium hexafluorophosphate. Commonly used side chain protecting groups are tert-butyl for N, Q, S and T; 2,2,4,6,7-pentametyldihydroxybenzofruan-5-sulfonyl for R; and butyloxycarbonyl for K. After synthesis, the peptides are deprotected and cleaved from the polymer support, e.g., by treatment with e.g., 92% trifluoracetic acid/4% triethylsilane/4% H2O. The peptides can be precipitated by the addition of tertbutylether/pentane (8:2) and purified by reversed-phase HPLC. The peptides are commonly analyzed by matrix-associated laser desorption time-of-flight mass spectrometry. By using these standard techniques, naturally occurring amino acids may also be substituted with unnatural amino acids such as D-stereoisomers, and also with amino acids with side chains having different lengths or functionalities. Functional groups for conjugating to small molecules, label moieties, peptides, or proteins may be introduced into the molecule during chemical synthesis. In addition, small molecules and label moieties may be attached during the synthetic process. Preferably, introduction of the functional groups and conjugation to other molecules minimally affects the structure and function of the subject peptide.

The N- and C-terminus of the peptides as well as any amino acid comprised in the peptide apart from the terminal amino acids may be derivatized using conventional chemical synthetic methods. The peptides of the invention may contain an acyl group, preferably C₁ to C₄ acyl such as an acetyl group. Methods for acylating, and specifically for acetylating the free amino group at the N-terminus are well known in the art. For the C-terminus, the carboxyl group may be modified by esterification with alcohols, preferably C₁ to C₄ alkanols, or amidated to form— CONH₂ or CONHR, R preferably being C₁ to C₄ alkyl. Methods of esterification and amidation are well known in the art.

The peptides of the invention may also be produced semi-synthetically, for example by a combination of recombinant and synthetic production. In the case that fragments of the peptide are produced synthetically, the remaining part of the peptides would have to be produced otherwise, e.g., recombinantly, and then be linked to the fragment to form the peptides of the invention. Recombinant production is described in EP 2 271 356.

The disclosure of EP 2 271 356 is incorporated by reference in its entirety.

The specific sequence of SEQ ID NO: 1 is embraced by the genus of peptides disclosed in EP 2 271 356. This document, however, fails to individualize this specific sequence, nor have its particularly outstanding antibiotic properties been recognized.

In a second aspect, the present invention provides a pharmaceutical composition comprising or consisting of a peptide in accordance with the first aspect.

In accordance with the present invention, the term “pharmaceutical composition” relates to a composition for administration to a patient, preferably a human patient. The pharmaceutical composition of the invention preferably comprises the peptide of the invention. It may, optionally, comprise further molecules capable of altering the characteristics of the peptide of the invention thereby, for example, stabilizing, modulating and/or activating its function. The composition may be in solid, liquid or gaseous form and may be, inter alia, in the form of (a) powder(s), (a) tablet(s), (a) solution(s) or (an) aerosol(s). The pharmaceutical composition of the present invention may, optionally and additionally, comprise a pharmaceutically acceptable carrier. By “pharmaceutically acceptable carrier” is meant a non-toxic solid, semi-solid or liquid filler, diluent, encapsulating material or formulation auxiliary or excipient of any type. Examples of suitable pharmaceutical carriers are well known in the art and include phosphate buffered saline solutions, water, emulsions, such as oil/water emulsions, various types of wetting agents, sterile solutions, organic solutions, organic solvents including DMSO. Compositions comprising such carriers can be formulated by well known conventional methods.

The pharmaceutical compositions can be administered to the subject at a suitable dose. The dosage regimen will be determined by the attending physician and clinical factors. As is well known in the medical arts, dosages for any one patient depends upon many factors, including the patient's size, body surface area, age, the particular compound to be administered, sex, time and route of administration, general health, and other drugs being administered concurrently. The therapeutically effective amount for a given situation will readily be determined by routine experimentation and is within the skills and judgment of the ordinary clinician or physician. Generally, the regimen as a regular administration of the pharmaceutical composition should be in the range of 1 μg to 5 g units per day. However, a more preferred dosage might be in the range of 0.01 mg to 100 mg, even more preferably 0.01 mg to 50 mg and most preferably 0.01 mg to 10 mg per day.

The pharmaceutical composition of the present invention may be administered systemically, topically or parenterally. The term “parenteral” as used herein refers to modes of administration which include intravenous, intramuscular, intraperitoneal, intrasternal, subcutaneous and intraarticular injection and infusion. Topical routes of administration include dermal, nasal and via inhalation.

In a third aspect, the present invention provides a peptide consisting of or comprising 17 to 23 amino acids, wherein the amino acids in positions 1 to 23, counted from the N-terminus, are as follows (1) G, S or lacking; (2) C or lacking; (3) K or R; (4) K or R; (5) Y, W or F; (6) K or R; (7) K or R; (8) F, W or L; (9) K or R; (10) K or L or lacking; (11) W, L or F; (12) K or R; (13) F, Y or C; (14) K or R; (15) G or Q; (16) K or R; (17) F, L or W; (18) F or W; (19) F, L or W; (20) W or F; (21) C or lacking; (22) F or G or lacking; (23) G or lacking, for use in a method of (a) treating, ameliorating or preventing a bacterial infection of the skin; (b) treating, ameliorating or preventing a bacterial wound infection; and/or (c) promoting wound healing.

The definition of the peptide in accordance with the third aspect embraces the specific peptide sequence in accordance with the first aspect. It is understood that, if not indicated explicitly to the contrary, peptides in accordance with any aspect of the present invention are linear and non-cyclic.

The present invention for the first time makes the above class of peptides available for the specific indications in accordance with items (a), (b) and (c), namely bacterial infections of the skin, bacterial wound infections and furthermore for the purpose of promoting wound healing. This is based on the present inventors' surprising finding that peptides in accordance with the third aspect, including the peptide of SEQ ID NO: 2 (as disclosed further below), allowed to combat bacterial infections of a badly closing wound where numerous attempts of established antibiotic therapy failed. Accordingly, not only is the prior art silent about usefulness of the peptides in accordance with the third aspect for the purposes in accordance with the third aspect, but furthermore are said peptides characterized by outstanding properties. In the view of the fact that any other antibiotic treatment of the mentioned badly healing wound failed, it has to be concluded that the peptides in accordance with the third aspect are in fact unique when compared to what has been previously available in terms of treatment options. Reference is made in particular to Example 5 in that respect.

The following preferred embodiments of the third aspect relate to preferred peptides, preferred bacteria, preferred medical indications, and envisaged underlying mechanisms.

Accordingly, in a preferred embodiment, (a) said peptide (i) is a peptide in accordance with the first aspect; or (ii) comprises or consists of the sequence of SEQ ID NO: 2 (GCKKYRRFRWKFKGKFWFWG); and/or (b) said use is topical.

The peptide consisting of the sequence of SEQ ID NO: 2 is also known as peptide 19-2.5 or Aspidasept®.

The term “topical” has its art-established meaning and refers to an administration of a peptide in accordance with the third aspect to the area affected by the bacterial infection and/or the area where wound healing is to be promoted and/or the proximity of the respective areas.

Suitable formulations for topical applications include ointments, solutions and sprays. Each of these formulations may comprise one or more pharmaceutically acceptable carriers, diluents or excipients. Compounds and compositions suitable as carriers, diluents and excipients are well known to the skilled person, described above, and available from a variety of manufacturers.

Furthermore, the formulations for topical application may comprise a deliver enhancer. Delivery enhancers are known in the art; see, e.g. WO 2011/064316.

In a further preferred embodiment, said bacterial infection of the skin or said bacterial wound infection is an infection by Gram positive and/or Gram negative bacteria, preferably by one or more of Staphylococcus such as Staphylococcus aureus including MRSA, β-hemolytic Streptococcus including group A Streptococcus such as Streptococcus pyogenes, Parvimonas including Parvimonas micra, Pseudomonas such as Pseudomonas aeruginosa including MDRPA, Haemophilus including Haemophilus influenza, Clostridium including Clostridium difficile, Enterococcus including vancomycin-resistant Enterococcus (VRE), Porphyromonas including Porphyromonas gingivalis, Propionibacterium including Propionibacterium acne and Acinetobacter including Acinetobacter baumannii.

The peptides of the present invention are useful with respect to combating in particular multi-resistant strains, independently of the kind of resistance mechanism, see Examples 1, 2 and 4.

In a further preferred embodiment, said bacterial infection of the skin or said bacterial wound infection is selected from erysipelas, impetigo, folliculitis, boil, cellulitis and carbuncle and/or is a nosocomial infection.

The above-mentioned specific indications are known in the art. In brief, erysipelas is an acute infection of the skin which predominantly affects the upper dermis and superficial lymphatics. Typical bacteria are group A Streptococcus, in particular Streptococcus pyogenes. Non-group A Streptococci may also be causative agents, but also rapidly growing mycobacteria, such as M. fortuitum, chelonae-abscessus group. For example, Streptococcus agalactiae is a non-group A Streptococcus. Cellulitis, which is also a bacterial infection, affects the inner layers of the skin, especially the dermis and the subcutaneous fat. A typical bacterium is Staphylococcus aureus. Of particular concern are the antibiotic resistant forms thereof such as methicillin-resistant Staphylococcus aureus (MRSA). Folliculitis is an infection of the hair follicles. Relevant bacterial species includes Staphylococcus aureus and Pseudomonas aeruginosa. Impetigo, in particular impetigo contagiosa, is a highly infectious disease of the skin which predominantly occurs in children and newborn. Typical bacteria are Staphylococcus aureus and Streptococcus pyogenes.

In terms of mechanism, it is envisaged that said peptide (a) inactivates the bacteria causing said infection; (b) binds and/or inactivates the toxins produced the bacteria causing said infection; and/or (c) acts anti-inflammatory.

Inactivation of bacteria includes a slowing down of bacterial growth, inhibition of bacterial growth, reduction of bacterial viability, partial killing of bacteria, and complete killing of bacteria.

The peptides in accordance with the invention are synthetic anti-lipopolysaccharide peptides (SALPs). Lipopolysaccharides (LPS) are comprised in the exterior membrane of Gram-negative bacteria. They function as antigens. Decay products thereof are released when bacteria die. These decay products are also known as endotoxins. Endotoxins contribute to the symptoms of a bacterial infection. The SALPs in accordance with the invention bind with high affinity to LPS and endotoxins. This significantly contributes to the beneficial effects of the peptides. In other words, the effect of endotoxins is neutralized by the peptides of the invention.

In a fourth aspect, the present invention relates to a pharmaceutical composition comprising or consisting of a combination of (a) two or more peptides, each peptide consisting of or comprising 17 to 23 amino acids, wherein the amino acids in positions 1 to 23, counted from the N-terminus, are as follows (1) G, S or lacking; (2) C or lacking; (3) K or R; (4) K or R; (5) Y, W or F; (6) K or R; (7) K or R; (8) F, W or L; (9) K or R; (10) K or L or lacking; (11) W, L or F; (12) K or R; (13) F, Y or C; (14) K or R; (15) G or Q; (16) K or R; (17) F, L or W; (18) F or W; (19) F, L or W; (20) W or F; (21) C or lacking; (22) F or G or lacking; (23) G or lacking; or (b) one or more peptides, each peptide consisting of or comprising 17 to 23 amino acids, wherein the amino acids in positions 1 to 23, counted from the N-terminus, are as follows (1) G, S or lacking; (2) C or lacking; (3) K or R; (4) K or R; (5) Y, W or F; (6) K or R; (7) K or R; (8) F, W or L; (9) K or R; (10) K or L or lacking; (11) W, L or F; (12) K or R; (13) F, Y or C; (14) K or R; (15) G or Q; (16) K or R; (17) F, L or W; (18) F or W; (19) F, L or W; (20) W or F; (21) C or lacking; (22) F or G or lacking; (23) G or lacking, and one or more antibiotics selected from small organic molecule antibiotics such as ceftriaxone, oxacillin, amoxicillin, amikacin, ciprofloxacin, erythromycin, imipenem and tetracycline, and peptidic antibiotics such as daptomycin and vancomycin.

This aspect of the invention relates to binary, ternary or higher order combinations of peptides or peptides with known antibiotics.

It is noted that certain peptides falling under the terms of the generic definition provided in part (a) of the fourth aspect as well as the antibiotics recited in the second part of item (b) of the fourth aspect are known. What was entirely unexpected, though, is the performance afforded by combinations as defined in accordance with the fourth aspect. The examples enclosed herewith contain evidence of very good performance for a plurality of combinations meeting the terms of the fourth aspect. The difference between combinations and individual agents is not only astonishing in a quantitative sense as evidenced by art-established measures of synergistic effect, but also in qualitative terms. For example, while individual agents merely reduce bacterial growth, combinations in accordance with the fourth aspect are capable of entirely abolishing bacterial growth.

In a preferred embodiment of the fourth aspect, (a) said combination is synergistic, and wherein synergism preferably occurs with regard to antibacterial activity; and/or (b) said pharmaceutical composition is a broad-spectrum antibiotic.

Antibacterial activity can be determined by any of the art-established measures. For example, for bacterial growth and suspension, photometric quantification may be used. This has been done, for example, in Example 2. Also, antibacterial activity may be determined via the number of colony-forming units (CFU) in a nutrient.

The combinations in accordance with the first aspect are not only characterized by outstanding performance and a high degree of synergistic activity, but furthermore in that they are active against a large number of bacteria. In that sense, the combinations in accordance with the fourth aspect are broad-spectrum antibiotics. As a consequence, the combinations in accordance with the fourth aspect are the first choice, especially in those cases where diagnosis, e.g., determining which bacteria are responsible for a given infection, is cumbersome or takes too long. This applies, for example, to sepsis. The examples enclosed herewith contain evidence of activity of combinations in accordance with the invention against the plurality of highly problematic bacteria. In particular, the evidence relates to multi-resistant E. coli ESBL (extended spectrum beta lactamase) as shown in Example 1, multi-resistant Staphylococcus aureus (MRSA) as shown in Example 2, and multi-resistant Pseudomonas aeruginosa PS-4.

To assess the synergy between peptides, the Fractional Inhibitory Concentration (FIC) index of each combination was calculated according to the following formula: [(A)/MICA]+[(P)/MICP]=FICA+FICP=FIC index where MICA and MICP are the MICs of the two agents determined separately. The same applies mutatis mutandis to ternary or higher order combinations. Values below 1 indicate synergy, a value of 1 indicates additivity, and values greater than 1 indicate antagonism.

Surprisingly, it has been found that even sub-inhibitory concentrations of peptides in accordance with the present invention provide for a synergistic effect with art-established antibiotics such as gentamycin, levofloxacin or oxacillin. Without wishing to be bound by a specific theory, it is considered that said sub-inhibitory concentrations (as well as higher concentrations) induce a massive entry of antibiotics into the bacterial cells.

In a further preferred embodiment of the fourth aspect, said combination is (a) a binary combination of (i) a peptide comprising or consisting of the sequence of SEQ ID NO: 1 and a peptide comprising or consisting of the sequence of SEQ ID NO: 2; (ii) a peptide comprising or consisting of the sequence of SEQ ID NO: 1 and an antibiotic as defined in accordance with the fourth aspect; or (iii) a peptide comprising or consisting of the sequence of SEQ ID NO: 2 and an antibiotic as defined in accordance with the fourth aspect; or (b) a ternary combination of a peptide comprising or consisting of the sequence of SEQ ID NO: 1, a peptide comprising or consisting of the sequence of SEQ ID NO: 2 and an antibiotic as defined in accordance with the fourth aspect.

Among the antibiotics which may be combined with peptides in accordance with the present invention, ceftriaxone and oxacillin are particularly preferred.

The peptides comprising or consisting of the sequences of SEQ ID NOs: 1 and 2, respectively, are particularly preferred agents in accordance with the present invention.

In a further preferred embodiment, (a) said pharmaceutical composition comprises a pharmaceutically acceptable carrier, diluent, or excipient; and/or (b) the recited pharmaceutically active agents are the only pharmaceutically active agents comprised in said pharmaceutical composition.

As common in the art, pharmaceutical compositions in accordance with the present invention may contain, in addition to pharmaceutically active agents, also pharmaceutically inactive agents including carriers, diluents and/or excipients. Exemplary carriers, diluents and excipients are described herein above.

Furthermore, it is preferred that no further pharmaceutically active agents beyond those explicitly recited are present in the pharmaceutical composition. Having said that, it is also envisaged that further, not explicitly mentioned pharmaceutically active agents are present. Among those further not explicitly mentioned agents, there is a preference for antibiotics.

In a fifth aspect, the present invention provides a pharmaceutical composition of any of the preceding claims for use in (a) a method of treating, ameliorating or preventing one or more conditions selected from sepsis, bacterial infections of the respiratory tract, bacterial infections of the gastrointestinal tract, bacterial infections of the urogenital tract, necrotizing fasciitis, bacterial infections of burns, bacterial wound infections, and bacterial infections of the skin; or (b) a method of promoting wound healing.

As noted above, sepsis is a particularly preferred indication in accordance with the present invention. The combinations, owing to their high antibiotic activity against a broad spectrum of bacteria are the first choice where rapid action against the diseases required which otherwise would become uncontrollable. As such, combinations of the invention are useful in the treatment of systemic infections. Having said that, also non-systemic disorders are among the indications amenable to treatment.

With regard to promoting wound healing in accordance with above item (b), it is noted that independent of the presence of bacteria or their toxins, the peptides of the invention are able to stimulate metalloproteinases such as the ADAMs and are able to recruit growth factors like the epidermal growth factor EGF.

In preferred embodiments of the fifth aspect, (a) the bacterial infection of the respiratory tract is tuberculosis, cystic fibrosis or COPD; (b) the bacterial infection of the gastrointestinal tract is Morbus Crohn; or (c) said condition is caused by Gram positive and/or Gram negative bacteria, especially by one or more of Staphylococcus such as Staphylococcus aureus including MRSA, Mycobacterium such as Mycobacterium tuberculosis including MDR and XDR strains, Pseudomonas such as Pseudomonas aeruginosa including MDRPA, Enterococcus including vancomycin-resistant Enterococcus (VRE), Haemophilus including Haemophilus influenzae, E. coli including ESBL, Klebsiella including Klebsiella pneumonia, β-hemolytic Streptococcus including group A Streptococcus such as Streptococcus pyogenes, and Acinetobacter including Acinetobacter baumannii.

The further aspects in accordance with the present invention as described below relate to the finding that individual peptides as well as combinations in accordance with the present invention are useful agents against biofilms. Biofilms are layers of bacteria on various types of surfaces, especially in hospitals and/or on devices which layers of bacteria are highly undesirable. The examples enclosed herewith contain evidence that agents in accordance with the present invention are capable of significantly reducing biofilms.

Accordingly, in a further aspect, the present invention provides a method of preventing or reducing formation of a biofilm on a device for intracorporeal use or on a surface in a hospital and/or of removing of a biofilm from a device for intracorporeal use or from a surface in a hospital, wherein said device is not present in a human or animal body, said method comprising bringing said device or surface into contact with a pharmaceutical composition as defined above or a peptide as defined above.

The term “biofilm” is known in the art and refers to a mucus layer with microorganisms embedded therein. Biofilms are formed by microorganisms at surfaces and adhere to the surface. In other words, a biofilm may also be referred to as a multicellular surface bound aggregate. Microorganisms may comprise or consist of bacteria of one species or a plurality of species.

Prevention or reduction of biofilm formation and removal of a biofilm may, depending on the case, be a cosmetic or medical application. Any method of prevention, reduction or removal may comprise further measures such as scrubbing.

In a further aspect, the present invention provides a method of preparing an intracorporeal device or a surface in a hospital, said method comprising bringing said device or surface into contact with a pharmaceutical composition as defined above or a peptide as defined above, wherein said device is not present in a human or animal body.

The term “preparing” as used in the context with this aspect of the invention relates to a conditioning or processing of the intracorporeal device or surface, respectively, as opposed to the method of manufacturing. This is also apparent from the following explanation of the step of “bringing into contact”.

The recited “bringing into contact” may be effected such that a peptide or the pharmaceutical composition as defined above is adsorbed or absorbed by the device for intracorporeal use or the surface.

The above recited requirement that the intracorporeal device is not present in a human or animal body ensures that methods of treatment therapy of the human or animal body are not within the ambit of the respective aspect of the invention. To the extent methods of therapeutic treatment of the human or animal body are not excluded from patentability, this negative feature is dispensable.

In a further aspect, the present invention provides a use of a pharmaceutical composition as defined above or a peptide as defined above for preventing or reducing formation of a biofilm on a device for intracorporeal use or on a surface in a hospital and/or for removing of a biofilm from a device for intracorporeal use or surface in a hospital, wherein said device is not present in a human or animal body.

In a further aspect, the present invention provides an intracorporeal device or a surface in a hospital which is coated and/or loaded with a pharmaceutical composition as defined above or a peptide as defined above.

The term “coated” refers to a layer on the surface of the intracorporeal device or the surface in a hospital which comprises or consists of one or more peptides and/or pharmaceutical compositions according to the invention. Said layer may cover all or parts of the surface or intracorporeal device.

The term “loaded” refers to an intracorporeal device or surface, wherein said device or surface in its entirety or parts thereof are made of a material which comprises one or more peptides and/or pharmaceutical compositions as defined herein above.

In preferred embodiments of the aspects of the invention relating to the control of biofilms, (a) said device for intracorporeal use is selected from catheters, implants, endoscopes, drainages, contact lenses and hearing aids; or (b) said surface in a hospital is a fitting.

In a further preferred embodiment, said biofilm comprises or consists of Gram positive or Gram-negative bacteria, preferably Pseudomonas aeruginosa or Staphylococcus aureus including MRSA.

In a further aspect, the present invention provides a nucleic acid encoding a peptide comprising or consisting of the sequence of SEQ ID NO: 1.

The term “nucleic acid” is used interchangeably with the term “polynucleotide” in accordance with the present invention and includes DNA, such as cDNA or genomic DNA, and RNA. Further included are nucleic acid mimicking molecules known in the art such as synthetic semi-synthetic derivatives of DNA or RNA and mixed polymers. Such nucleic acid mimicking molecules or nucleic acid derivatives according to the invention include phosphorothiotate nucleic acid, phosphoramidate nucleic acid, 2′-O-methoxyethyl ribonucleic acid, morpholino nucleic acid, hexitol nucleic acid (HNA) and locked nucleic acid (LNA) (see Braasch and Corey, Chem. Biol. 2001, 8: 1). LNA is an RNA derivative in which the ribose ring is constrained by a methylene linkage between the 2′-oxygen and the 4′-carbon. They may contain additional non-natural or derivative nucleotide bases, as will be readily appreciated by those skilled in the art. For the purposes of the present invention, also a peptide nucleic acid (PNA) can be used. Peptide nucleic acids have a backbone composed of repeating N-(2-aminoethyl)-glycine units linked by peptide bonds. The purine and pyrimidine bases are linked to the backbone by methylene carbonyl bonds.

In a preferred embodiment, the nucleic acid molecule is DNA.

It will be readily appreciated by the skilled person that more than one nucleic acid may encode the peptides of the present invention due to the degeneracy of the genetic code. Degeneracy results because a triplet base code composed of four bases designates each of the 20 proteinogenic amino acids and a stop codon. The possible 43 possibilities for bases in triplets gives 64 possible codons, meaning that some degeneracy must exist. As a result, some amino acids are encoded by more than one triplet, i.e., by up to six. The degeneracy mostly arises from alterations in the third position in a triplet. This means that nucleic acid molecules having different sequences, but still encoding the same polypeptide lie within the scope of the present invention.

In a further aspect, the present invention provides a vector comprising the nucleic acid as defined above.

Preferably, the vector is a plasmid, cosmid, virus, bacteriophage or another vector used conventionally e.g., in genetic engineering.

Preferably, the vector is an expression vector.

An expression vector according to this invention is capable of directing the replication, and the expression of the nucleic acid molecule of the invention and the peptide encoded thereby. Suitable expression vectors are described above.

In a further aspect, the present invention provides an in vitro method of controlling the growth of bacteria, wherein said method comprises bringing into contact said bacteria with a peptide as defined above or with a combination as defined above.

In a further aspect, the present invention provides a of a peptide as defined above or a combination as defined above for controlling the growth of bacteria.

In preferred embodiments, said controlling comprises or consists of reducing, slowing down, inhibiting or abolishing.

The Figures Show:

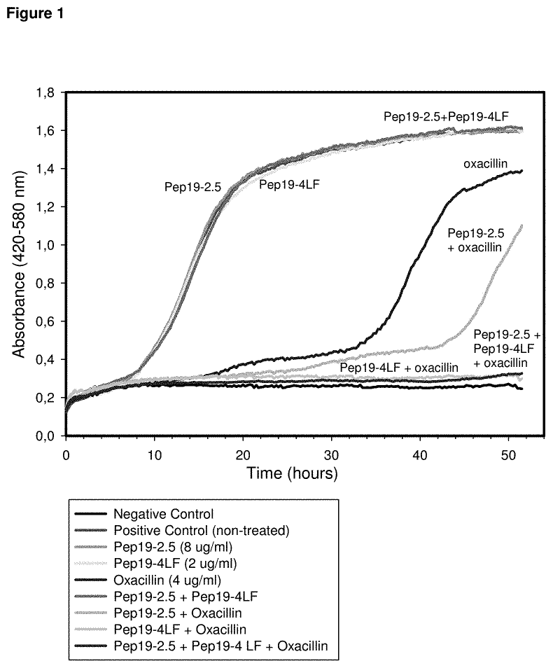

FIG. 1: Bacterial growth in presence and absence of agents of the invention.

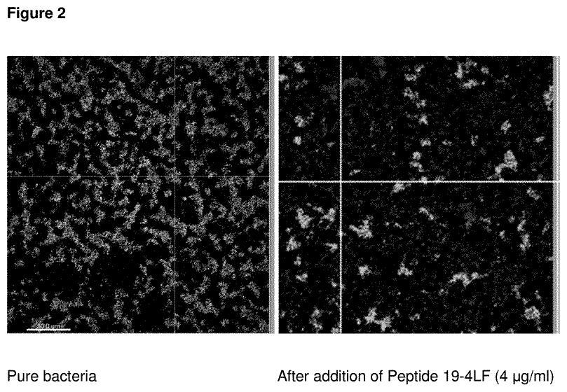

FIG. 2: Inactivation of a biofilm from Pseudomonas aeruginosa with GFP-labelled bacteria (GFP: Green-Fluorescent Protein). Living bacteria: white, inactivated bacteria: dark.

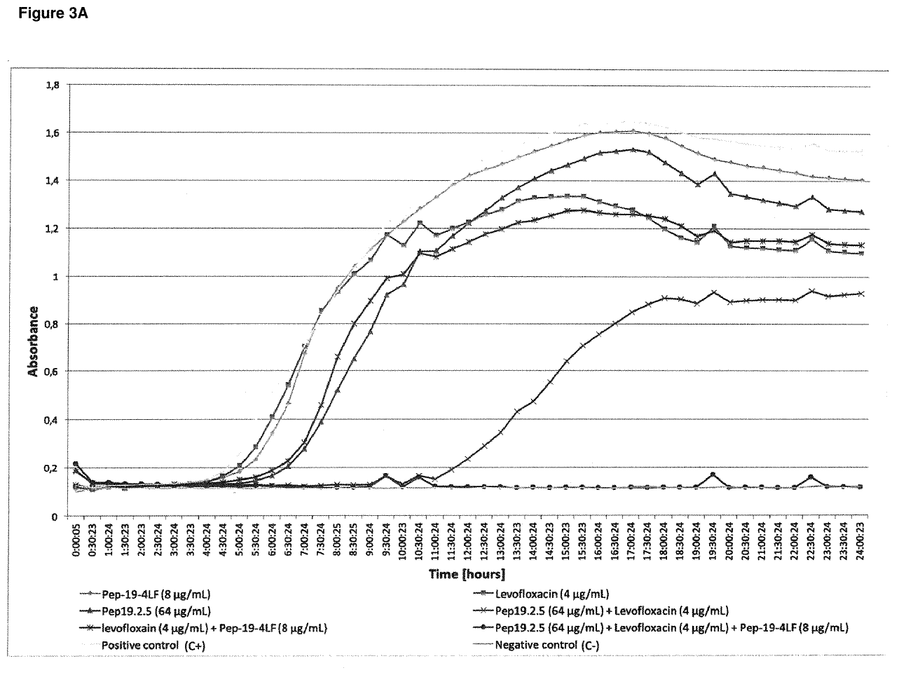

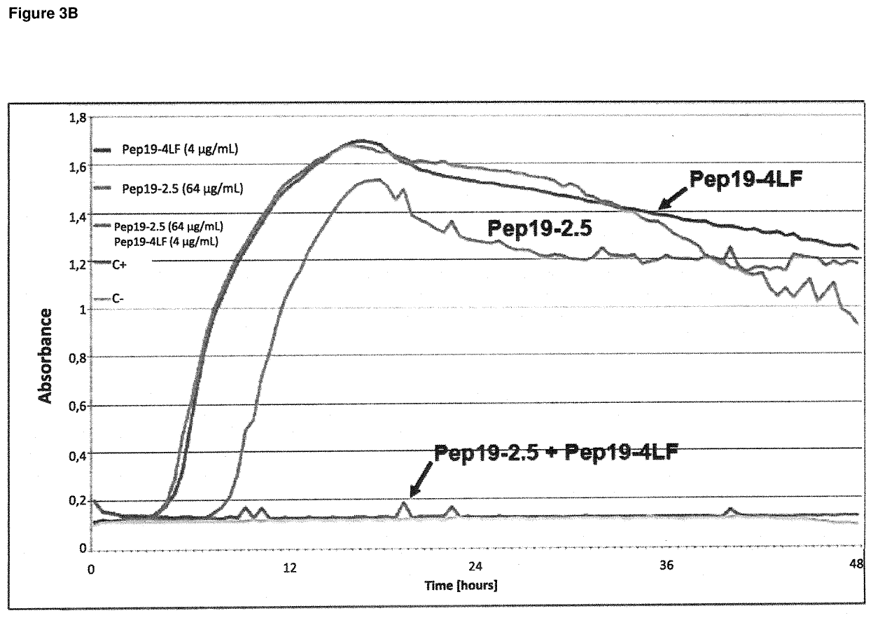

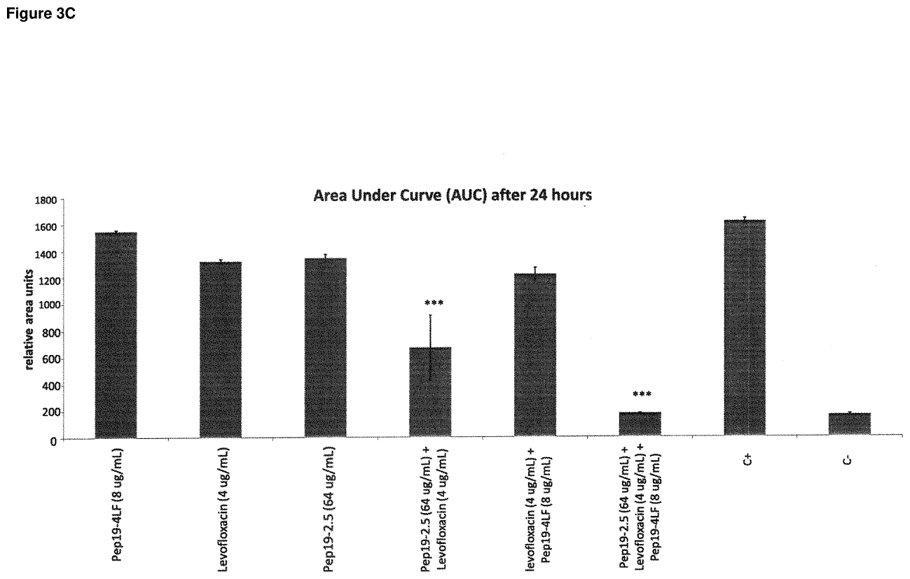

FIGS. 3A-3C: Peptide 19-2.5/Peptide 9-4LF/levofloxacin (3A). Peptide 19-2.5/Peptide 9-4LF/levofloxacin (3B). The area under the curve (AUC) is a measure of the antimicrobial effectiveness of the drugs, with lowest values exhibiting the strongest effects (3C).

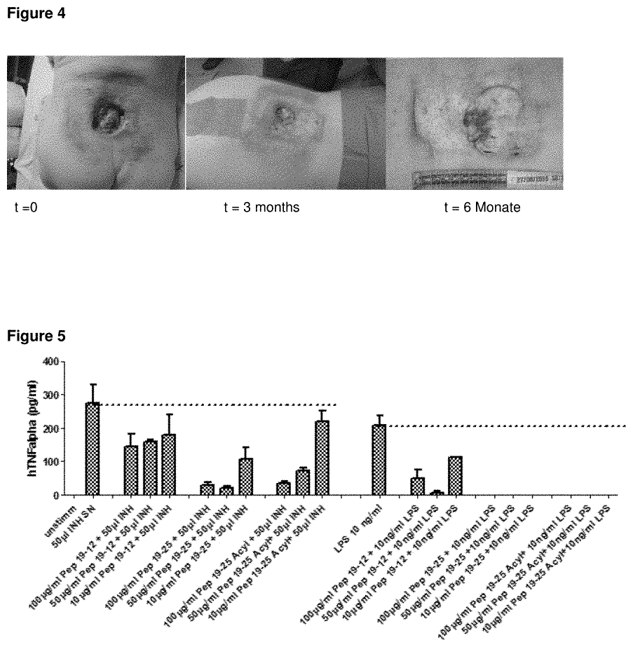

FIG. 4: Wound before and after daily treatment at t=0, 3 months and 6 months.

FIG. 5: Does dependent inhibition of M. tuberculosis-induced TNF formation of human peripheral blood mononuclear cells by Peptide 19.2.5. Supernatants of Isoniazid-treated M. tuberculosis bacteria (0,1 μg/ml; 72 h) were left untreated or incubated for 30 min with the peptides X, Y, Z at the concentrations indicated. Equal amounts were subsequently added to human PBMC (1×10⁶/ml) and incubated for further 24 h. The TNF formation was measured by ELISA (R&D Systems).

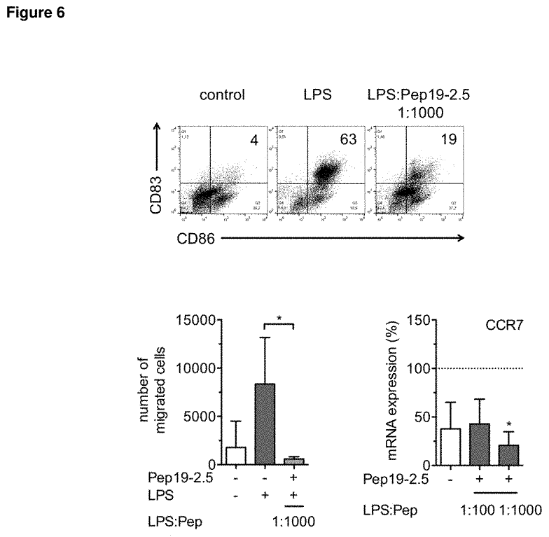

FIG. 6: Aspidasept® inhibits maturation and migration of MoDCs.

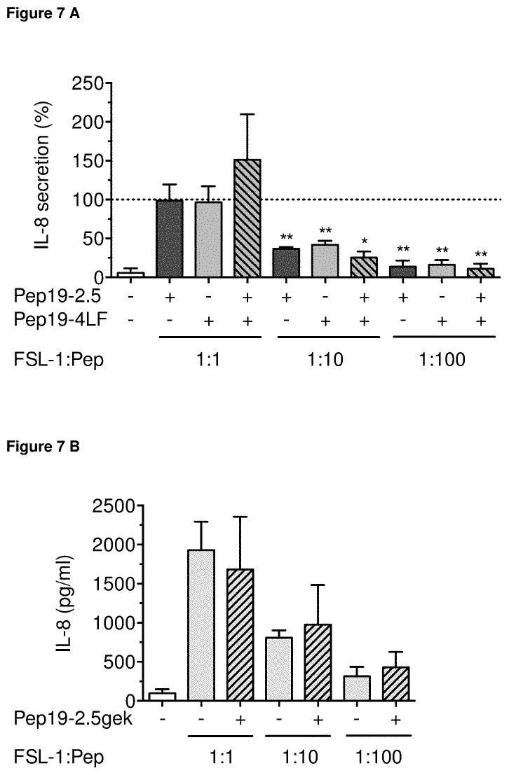

FIGS. 7A-7B: Aspidasept® (FIG. 7A, Pep19-2.5) and Aspisasept II (FIG. 7A, Pep 19-4LF) reduce IL-8 release in TLR2/6-activated keratinocytes, whereas the control peptide Pep19-2.5gek was completely inactive (FIG. 7B).

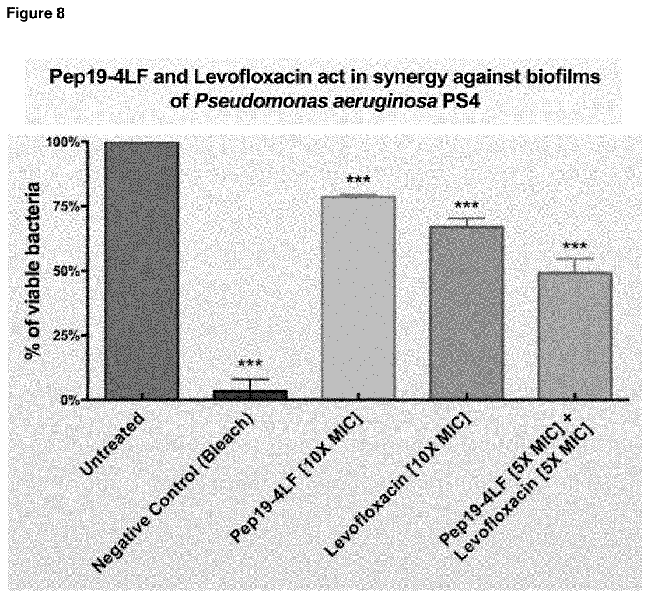

FIG. 8: Inhibition of biofilm formation by Aspisasept II (Pep 19-4LF) with and without levofloxacin.

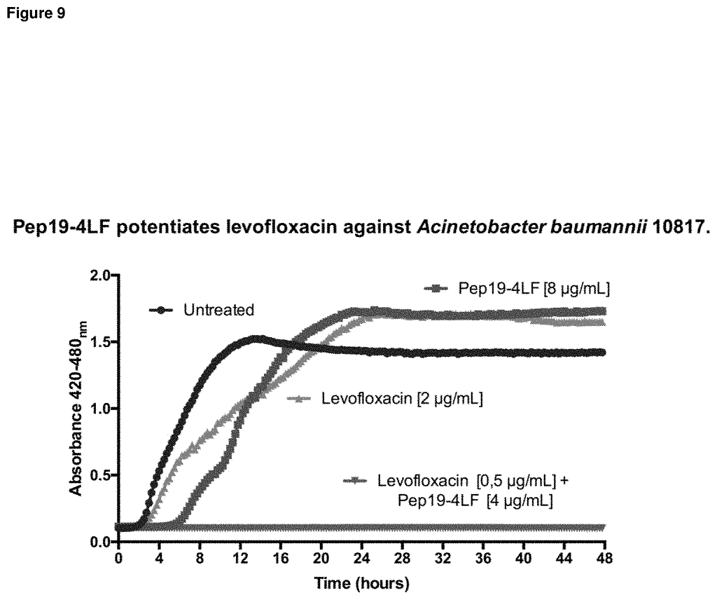

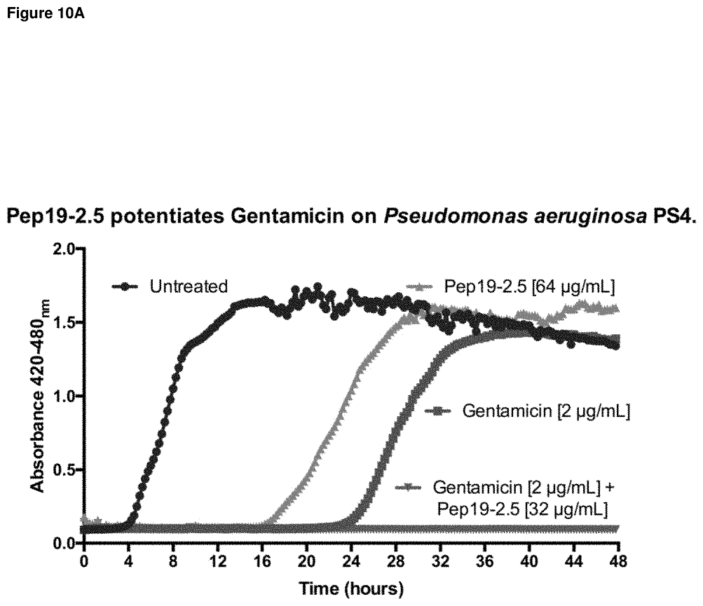

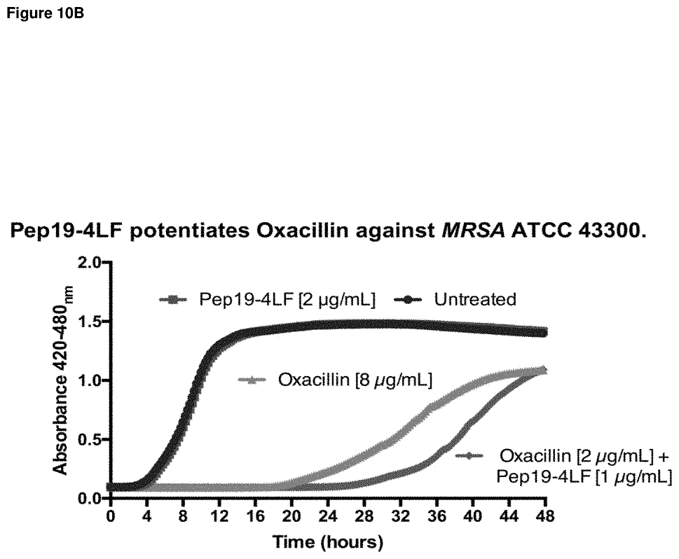

FIG. 9: Synergistic effect of the combination of levofloxacin with a peptide of the invention. Data were obtained from growth experiments in Mueller-Hinton (cation adjusted) with continuous shaking at 37° C. using Bioscreen C, Labsystem, Helsinki, Finland. Concentrations were adjusted after checkerboard analysis, once Fractional Inhibitory Concentrations (FIC) were calculated. All cases resulted in a FIC index <0,5, indicating synergistic effect. Results show the average of three independent experiments.

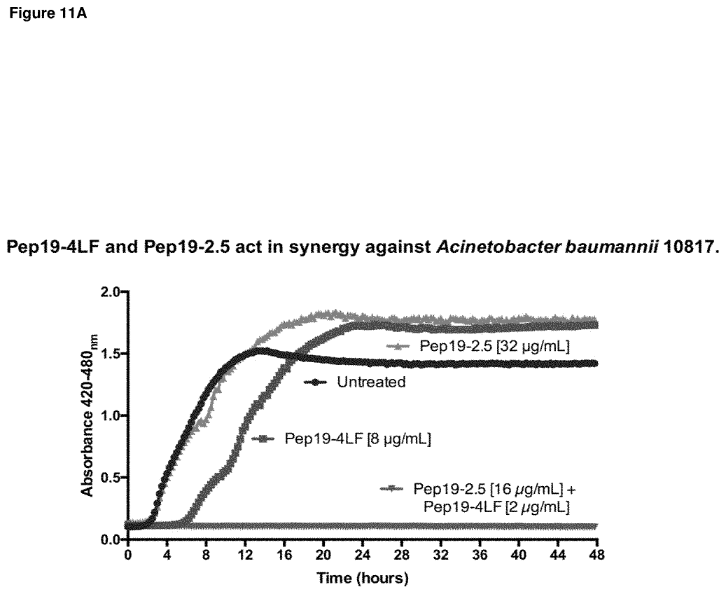

FIGS. 10A-10B: Synergistic effect of the combination of gentamycin with Aspidasept® (Pep19-2.5) (10A) and the combination of oxacillin with Aspisasept II (Pep 19-4LF) (10B), respectively.

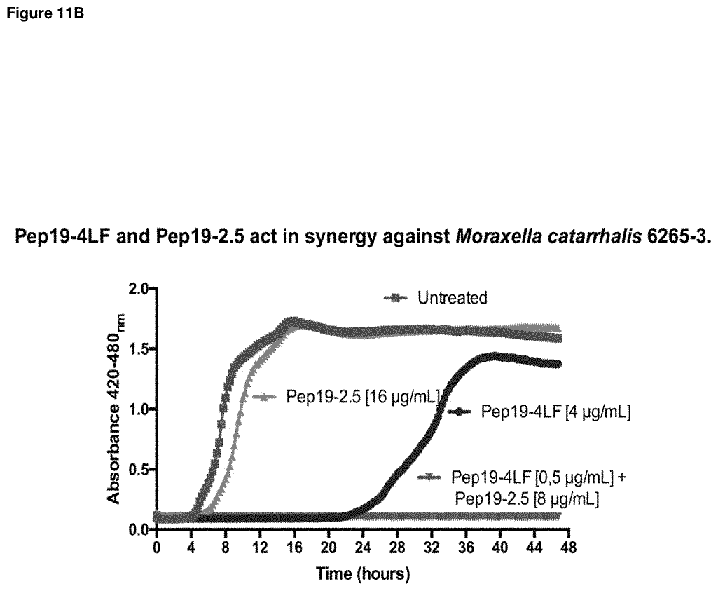

FIGS. 11A-11B: Synergistic effect of the combination of two peptides of the invention (Aspidasept® and Aspidasept II) against Acinetobacter baumannii (FIG. 11A) and Moraxella catarrhalis (FIG. 11B).

The Examples illustrate the invention.

### Example 1

### Growth of Escherichia coli ESBL (CUN E20) in the Presence of Combinations of Ceftriaxon and Peptides of the Invention

The ability of the peptides to induce bacterial sensitization to antibiotics was determined by a standard checkerboard titration method in 96-well polystyrene microtiter plates [Eliopoulos GM, Moellering, RC: Antimicrobial combinations. In Antibiotics in Laboratory Medicine 4th edition. Edited by: Lorian V. Baltimore: The Williams and Wilkins Co; 1996:330-396]. For this purpose, serial dilutions of the two antimicrobial agents were mixed together in a microtiter plate so that each row contained a fixed amount of one agent and increasing amounts of the other. Inocula consisted of 1×10⁵ CFU/mL, approximately in Mueller-Hinton (MH) medium (Difco Laboratories, Sparks, MD, USA). To assess the synergy between peptides, the Fractional Inhibitory Concentration (FIC) index of each combination was calculated according to the following formula: [(A)/MICA]+[(P)/MICP]=FICA+FICP=FIC index, where MICA and MICP are the MICs of the two agents determined separately, and (A) and (P) are the MICs of the agents when determined in combination. A given peptide-peptide combination was considered as synergistic if its FIC index was 0.5. Peptides with a MIC higher than the maximum concentration tested (250 μg/mL) were arbitrarily assigned a MIC value of 500 μg/mL.

TABLE 1

| | | | | | | | | | | | |
|---|---|---|---|---|---|---|---|---|---|---|---|
| CFX 2 | CFX 2 | CFX 2 | CFX 2 | CFX 2 | CFX 2 | CFX 2 | CFX 2 | CFX 2 | CFX 2 | 4LF 8 | Positive Control |
| P19-2.5 16 | P19-2.5 8 | P19-2.5 4 | P19-2.5 2 | P19-2.5 1 | P19-2.5 0.5 | 19-2.5 0.25 | 19-2.5 0.12 | 19-2.5 0.06 | 19-2.5 0.03 | | of Growth (no |
| 4LF 8 | 4LF 8 | 4LF 8 | 4LF 8 | 4LF 8 | 4LF 8 | 4LF 8 | 4LF 8 | 4LF 8 | 4LF 8 | | Antimicrob.) |
| CFX 2 | CFX 2 | CFX 2 | CFX 2 | CFX 2 | CFX 2 | CFX 2 | CFX 2 | CFX 2 | CFX 2 | 4LF 4 | Positive |
| P19-2.5 16 | P19-2.5 8 | P19-2.5 4 | P19-2.5 2 | P19-2.5 1 | P19-2.5 0.5 | 19-2.5 0.25 | 19-2.5 0.12 | 19-2.5 0.06 | 19-2.5 0.03 | | Control of |
| 4LF 4 | 4LF 4 | 4LF 4 | 4LF 4 | 4LF 4 | 4LF 4 | 4LF 4 | 4LF 4 | 4LF 4 | 4LF 4 | | Growth (no |
| | | | | | | | | | | | Antimicrob.) |
| CFX 2 | CFX 2 | CFX 2 | CFX 2 | CFX 2 | CFX 2 | CFX 2 | CFX 2 | CFX 2 | CFX 2 | 4LF 2 | Positive Control |
| P19-2.5 16 | P19-2.5 8 | P19-2.5 4 | P19-2.5 2 | P19-2.5 1 | P19-2.5 0.5 | 19-2.5 0.25 | 19-2.5 0.12 | 19-2.5 0.06 | 19-2.5 0.03 | | of Growth (no |
| 4LF 2 | 4LF 2 | 4LF 2 | 4LF 2 | 4LF 2 | 4LF 2 | 4LF 2 | 4LF 2 | 4LF 2 | 4LF 2 | | Antimicrob.) |
| CFX 2 | CFX 2 | CFX 2 | CFX 2 | CFX 2 | CFX 2 | CFX 2 | CFX 2 | CFX 2 | CFX 2 | 4LF 1 | Positive Control |
| P19-2.5 16 | P19-2.5 8 | P19-2.5 4 | P19-2.5 2 | P19-2.5 1 | P19-2.5 0.5 | 19-2.5 0.25 | 19-2.5 0.12 | 19-2.5 0.06 | 19-2.5 0.03 | | of Growth (no |
| 4LF 1 | 4LF 1 | 4LF 1 | 4LF 1 | 4LF 1 | 4LF 1 | 4LF 1 | 4LF 1 | 4LF 1 | 4LF 1 | | Antimicrob.) |
| CFX 2 | CFX 2 | CFX 2 | CFX 2 | CFX 2 | CFX 2 | CFX 2 | CFX 2 | CFX 2 | CFX 2 | 4LF 0.5 | Positive Control |
| P19-2.5 16 | P19-2.5 8 | P19-2.5 4 | P19-2.5 2 | P19-2.5 1 | P19-2.5 0.5 | 19-2.5 0.25 | 19-2.5 0.12 | 19-2.5 0.06 | 19-2.5 0.03 | | of Growth (no |
| 4LF 0.5 | 4LF 0.5 | 4LF 0.5 | 4LF 0.5 | 4LF 0.5 | 4LF 0.5 | 4LF 0.5 | 4LF 0.5 | 4LF 0.5 | 4LF 0.5 | | Antimicrob.) |
| P19-2.5 16 | P19-2.5 8 | P19-2.5 4 | P19-2.5 2 | P19-2.5 1 | P19-2.5 0.5 | 19-2.5 0.25 | 19-2.5 0.12 | 19-2.5 0.06 | 19-2.5 0.03 | Sterility | Positive Control |
| 4LF 8 | 4LF 8 | 4LF 8 | 4LF 8 | 4LF 8 | 4LF 8 | 4LF 8 | 4LF 8 | 4LF 8 | 4LF 8 | control | of Growth (no |
| | | | | | | | | | | (No | Antimicrob.) |
| | | | | | | | | | | inoculum) | |
| P19-2.5 16 | P19-2.5 8 | P19-2.5 4 | P19-2.5 2 | P19-2.5 1 | P19-2.5 0.5 | 19-2.5 0.25 | 19-2.5 0.12 | 19-2.5 0.06 | 19-2.5 0.03 | Sterility | Positive Control |
| | | | | | | | | | | control | of Growth (no |
| | | | | | | | | | | (No | Antimicrob.) |
| | | | | | | | | | | inoculum) | |
| CFX 2 | CFX 2 | CFX 2 | CFX 2 | CFX 2 | CFX 2 | CFX 2 | CFX 2 | CFX 2 | CFX 2 | Sterility | Positive Control |
| P19-2.5 16 | P19-2.5 8 | P19-2.5 4 | P19-2.5 2 | P19-2.5 1 | P19-2.5 0.5 | 19-2.5 0.25 | 19-2.5 0.12 | 19-2.5 0.06 | 19-2.5 0.03 | control | of Growth (no |
| | | | | | | | | | | (No | Antimicrob.) |
| | | | | | | | | | | inoculum) | |

CFX: Ceftriaxone;
P19-2.5: Peptide 19-2.5;
4LF: Peptide 19-4LF;
Numbers are expressed in μg/mL.
Squares in grey and white correspond to microplate wells with and without growth, respectively.

The MIC values for the individual agents are as follows: Peptide 19-4LF: 16 μg/ml; Peptide 19-2.5: 128 μg/ml; Ceftriaxon: 16 μg/ml.

The index of Fractioned Inhibitory Concentration (FIC) is used as a measure of synergy. Double and triple synergy, respectively, were found for the following combinations:

Binary combination Cef+Peptide 19-2.5: FIC 0,375; binary combination Cef+Peptide 19-4LF: FIC 0.5; binary combination Peptide 19-4LF+Peptide 19-2.5: FIC 0.5; and ternary combination Cef+Peptide 19-2.5+Peptide 19-4LF: FIC 0.375.

### Example 2

### Synergistic Effects on Growth of MRSA

Growth curves of Methicillin Resistant Staphylococcus aureus ATCC 43300 (MRSA) exposed at time 0 to combinations of Pep 19-2.5 (8 μg/mL), Pep 19-4LF (2 μg/mL) and oxacillin (4 μg/mL) are shown in FIG. 1.

The inhibitory activity of combinations were determined by an automated turbidimetric-based system (Bioscreen C, Labsystem, Helsinki, Finland), which measures absorbance of the culture at regular intervals. Assays were performed in MH broth using Bioscreen polystyrene honeycomb 100-well plates. Inocula consisted of 1×10⁵ CFU/mL, approximately in Mueller-Hinton (MH) medium (Difco Laboratories, Sparks, MD, USA). Cell suspensions were grown at 37° C. with shaking (control set at “medium r.p.m.” position) and the absorbance was determined every 15 min for at least 48 h.

The absorbance of the cultures was measured every 15 minutes using an automated Bioscreen C system.

### Example 3

### Inactivation of Biofilm

The data shown in FIG. 2 indicate that already after short-time treatment (1 h) most bacteria are inactivated. The representative data shown in FIG. 2 indicated that after short-time of Peptide 19-4LF addition (1h) the most bacteria are inactivated. The micrographics shown that only a small fraction of the surface was covered by live bacteria forming a biofilm after 1 h (B) compared with the untreated one (A). The degree of reduction in life bacteria was very obvious, suggesting the Peptide 19-4LF can penetrate the extracellular polymeric substance (EPS) matrix and kill the bacteria after a short time period.

The data shown in FIG. 8 were obtained in experiments carried out using the Center for Disease Control (CDC) Biofilm Reactor (CBR). In this case, clinical strain Pseudomonas aeruginosa PS4 was incubated in TSB under continuous shaking for 24 hours, followed by additional 24 hours growth with a flow of diluted TSB. Samples received treatments during another 24 hours dissolved at Phosphate Buffer at 37° C. Biofilms were scraped and serially diluted prior to plating and counting. For confocal microscopy, Biofilms were stained using Live/Death BacLight (Life technologies, Carlsbad, California, USA). Results show the average of two independent experiments.

### Example 4

### Kinetics of Inhibitory Synergistic Action: Antibiotic Peptides of the Invention in Combination with Levofloxacin

Combinations of Peptide 19-2.5 and Peptide 19-4LF with the antibiotic levofloxacin (third generation drug from the group of fluorochinolones) and combinations of Peptide 19-2.5 and Peptide 19-4LF alone have been tested on multiresistant bacteria from Pseudomonas aeruginosa PS4.

In particular, the inhibitory activity of combinations were determined by an automated turbidimetric-based system (Bioscreen C, Labsystem, Helsinki, Finland), which measures absorbance of the culture at regular intervals. Assays were performed in MH broth using Bioscreen polystyrene honeycomb 100-well plates. Inocula consisted of 1×10⁵ CFU/mL, approximately in Mueller-Hinton (MH) medium (Difco Laboratories, Sparks, MD, USA). Cell suspensions were grown at 37° C. with shaking (control set at “medium r.p.m.” position) and the absorbance was determined every 15 min for at least 48 h. Each experiment was repeated three times independently and the results were analyzed with the Prism program. For this purpose, first the area under the curve was obtained for each triplicate and the average result was statistically analyzed using the nonparametric Mann-Whitney U supplemented with Kruskal Wallis test for pairwise comparisons.

In FIGS. 3 and 9, the growth of the bacteria (measured optically: optical density) is plotted versus time (in hours). Both combinations, the peptides with the antibiotic as well as the peptides alone are potent synergistic combinations, evidenced by FIC values below 0.5 (FIC=0.31 in FIG. 9).

The peptides alone as well as with antibiotics (beside levofloxacin also gentamycin) act synergistically against multi-resistant strains from Pseudomonas aeruginosa as well as from Acinetobacter baumanii.

Kinetics of inhibitory synergistic action: antibiotic peptides of the invention in combination with gentamycin and oxacillin, respectively.

The data shown in FIGS. 10A-10B were obtained in growth experiments in Mueller-Hinton (cation adjusted) with continuous shaking at 37° C. using Bioscreen C, Labsystem, Helsinki, Finland. Concentrations were adjusted after checkerboard analysis, once Fractional Inhibitory Concentrations (FIC) were calculated. All cases resulted in a FIC index <0,5, indicating synergistic effect. Results show the average of three independent experiments.

### Example 5

### Healing Attempt According to the German Arzneimittelgesetz § 4b with a Non-Approved Drug

A male patient (78 years old) had—due to a cured tumor in the back—an open wound (see picture at t=0). Because of the bad soft tissue conditions after radiotherapy, it was decided to perform an operative rehabilitation which, however, did not succeed. Therefore, a conservative wound treatment was initiated. The wound's localization required the involvement of an ambulant nursing service who took over the daily care. Nevertheless, in sporadic intervals, wound infections occurred which inhibited the healing process. A microbiological analysis showed the occurrence of Staphylococcus aureus, Parvimonas micra, β-hemolysing Streptococcus, and Pseudomonas aeruginosa. Over 6 years, all therapeutical approaches with different antibiotics and topically applied salve formulations failed. In November 2014 the patient was informed about the possibility of a, healing attempt′, to which he agreed. The therapy was started with 0.1% Pep19-2.5 (Aspidasept®) in salve (BACHEM Lot. 1053821 in DAC-base cream from pharmacist), which showed no effect. Only after increase of the concentration to 1% a significant effect was observed (see picture below at t=3 months in February 2015), connected with an increasing healing of the wound. Already after 2 months the diameter of the wound was reduced by 50%, and completely healed after 6 months; see FIG. 4. This therapeutically effect can be explained only from the use of the Aspidasept® salve, since all other parameters were not changed. The wound is now completely closed.

### Example 6

### Inhibition of the Inflammatory Response Induced by Cell-Wall Compounds of Mycobacterium tuberculosis

M. tuberculosis bacteria were treated with the anti-Mtb first line antibiotics isoniazid (INH) or rifampicin (RIF) for 3 days at 37° C. Subsequently, the supernatant of the bacterial cells was added to human mononuclear cells (5×10⁵ cells/a) in the absence or presence of the peptides Pep19-2.5, Pep19-12 and Pep19-2.5 Acyl (Hexanoic residue). The inflammatory response was monitored by measuring tumor-necrosis-factor α (TNFα) formation in an ELISA. It was observed that compound Pep19-2.5 exhibits the strongest anti-TNFα activity, already at a rather low concentration of 10 μg/ml the TNFα formation was inhibited (Figure on the left) by more than 50%. Other peptides with sequence variations showed a weaker inhibitory activity. As control, the inactivation of the LPS-induced TNFα production is presented for the three investigated peptides on the right side of the figure, with a very efficient inhibition as previously described by T. Gutsmann et al. (Gutsmann et al., New antiseptic peptides to protect against endotoxin-mediated shock, AAC 54, 3817-3824 (2010)). See FIG. 5.

### Example 7

### Aspidasept® Inhibits Maturation and Migration of MoDCs

Maturation and migration of immature dendritic cells are key steps in the initiation of adaptive immunity against pathogens. However, sustained and excessive inflammatory responses mediated by activated T cells may contribute to chronic inflammation and delayed wound healing.

In monocyte-derived dendritic cells (MoDCs), Pep19-2.5 inhibited LPS-mediated upregulation of the maturation marker CD83 and the co-stimulatory molecule CD80 at a peptide:LPS weight ratio of 1000:1. In addition, LPS-induced migration of MoDCs and CCR7 gene expression was completely blocked by Pep19-2.5; see FIG. 6.

### Example 8

### Aspidasept® and Aspidasept II Reduce IL-8 Release in TLR2/6-Activated Keratinocytes

It can be shown that the keratinocytes from human skin do not react to Gram-negative endotoxin (LPS), apparently due to the lack of the TLR4-receptor and the fact that most bacteria from the skin are of Gram-positive origin. Thus, the effect of Pep19-2.5 and Pep19-4LF on the toxin (pathogenicity factor) FSL-1 (fibroblast-stimulating lipopeptide), a TLR-2/6 activating compound, was checked.

It could be shown that the IL-8 inducing activity of FSL-1 was considerably reduced by the addition of the peptides already at a 10:1 weight ratio, whereas the control peptide Pep19-2.5gek, lacking the C-terminal end of the two peptides, was completely inactive; see FIGS. 7A-7B.

### Example 9

### Suppression of Inflammatory Responses in Skin Cells and Promotion of Keratinocyte Migration

The potential of Peptide 19-2.5 and the structurally related compound Peptide 19-4LF has been investigated for their therapeutic application in bacterial skin infections. Primary human keratinocytes responded to TLR2 (FSL-1) but not TLR4 (LPS) activation by increased IL-8 production which was determined with ELISA. Both SALPs inhibited FSL-1-induced phosphorylation of NF-κB p65 and p38 MAPK and significantly reduced IL-8 release and gene expression of IL-1β, CCL2 (MCP-1) and hBD-2. To detect phosphorylation of the intracellular proteins Western Blot was performed. Gene expression was evaluated by quantitative real-time PCR. In the MTT test cytotoxicity was observed at SALP concentrations below 10 μg/ml. In LPS-stimulated monocyte-derived dendritic cells, the peptides blocked IL-6 secretion, downregulated expression of the maturation markers CD83 and CD86— detected with flow cytometry—and inhibited CCR7-dependent migration capacity. Similarly, monocyte-derived Langerhans-like cells activated with LPS and pro-inflammatory cytokines showed reduced IL-6 levels and CD83/CD86 expression in the presence of SALPs. In addition to acute inflammation, bacterial infections often result in impaired wound healing. Since re-epithelialization is a critical step in wound repair, we tested whether Peptide 19-2.5 affects keratinocyte migration. In a scratch assay the peptide markedly promoted cell migration and accelerated artificial wound closure at concentrations as low as 1 ng/ml and was equipotent to TGF-β.

### Example 10

### Synergistic Action of Two Peptides of the Invention in Further Bacteria

FIGS. 11A-11B show data obtained in growth experiments in Mueller-Hinton (cation adjusted) with continuous shaking at 37° C. using Bioscreen C, Labsystem, Helsinki, Finland. Concentrations were adjusted after checkerboard analysis, once Fractional Inhibitory Concentrations (FIC) were calculated. All cases resulted in a FIC index <0,5, indicating synergistic effect. Results show the average of three independent experiments.

1. A pharmaceutical composition comprising or consisting of a combination of
(a) two or more peptides, each peptide consisting of or comprising 17 to 23 amino acids, wherein the amino acids in positions 1 to 23, counted from the N-terminus, are as follows (1) G, S or lacking; (2) C or lacking; (3) K or R; (4) K or R; (5) Y, W or F; (6) K or R; (7) K or R; (8) F, W or L; (9) K or R; (10) K or L or lacking; (11) W, L or F; (12) K or R; (13) F, Y or C; (14) K or R; (15) G or Q; (16) K or R; (17) F, L or W; (18) F or W; (19) F, L or W; (20) W or F; (21) C or lacking; (22) F or G or lacking; (23) G or lacking; or
(b) one or more peptides, each peptide consisting of or comprising 17 to 23 amino acids, wherein the amino acids in positions 1 to 23, counted from the N-terminus, are as follows (1) G, S or lacking; (2) C or lacking; (3) K or R; (4) K or R; (5) Y, W or F; (6) K or R; (7) K or R; (8) F, W or L; (9) K or R; (10) K or L or lacking; (11) W, L or F; (12) K or R; (13) F, Y or C; (14) K or R; (15) G or Q; (16) K or R; (17) F, L or W; (18) F or W; (19) F, L or W; (20) W or F; (21) C or lacking; (22) F or G or lacking; (23) G or lacking, and one or more antibiotic.

2. The pharmaceutical composition of claim 1, wherein the one or more antibiotics are small organic molecule antibiotics or peptidic antibiotics.

3. The pharmaceutical composition of claim 2, wherein the small organic molecule antibiotics are selected from the group consisting of ceftriaxone, oxacillin, amoxicillin, amikacin, ciprofloxacin, erythromycin, imipenem, and tetracycline.

4. The pharmaceutical composition of claim 2, wherein the peptidic antibiotics are selected from the group consisting of daptomycin and vancomycin.

5. The pharmaceutical composition of claim 1, wherein
(a) the combination is synergistic, and wherein synergism preferably occurs with regard to antibacterial activity; and/or
(b) the pharmaceutical composition includes a broad-spectrum antibiotic.

6. The pharmaceutical composition of claim 1, wherein the combination is a combination of
(i) a peptide comprising the sequence of SEQ ID NO: 1 and a peptide comprising the sequence of SEQ ID NO: 2;
(ii) a peptide comprising the sequence of SEQ ID NO: 1 and an antibiotic, wherein the antibiotic is a broad-spectrum antibiotic; or
(iii) a peptide comprising the sequence of SEQ ID NO: 2 and an antibiotic, wherein the antibiotic is a broad-spectrum antibiotic.

7. The pharmaceutical composition of claim 1, wherein the combination is a combination of a peptide comprising the sequence of SEQ ID NO: 1, a peptide comprising the sequence of SEQ ID NO: 2 and a broad-spectrum antibiotic.

8. A method for treating or ameliorating one or more conditions selected from sepsis, bacterial infections of the respiratory tract, bacterial infections of the gastrointestinal tract, bacterial infections of the urogenital tract, necrotizing fasciitis, bacterial infections of burns, bacterial wound infections, and bacterial infections of the skin, comprising administering the pharmaceutical composition of claim 1 to a patient in thereof.

9. A method for promoting wound healing comprising administering the pharmaceutical composition of claim 1 to a patient in need thereof.

10. The method of claim 8, wherein the bacterial infection of the respiratory tract causes tuberculosis, cystic fibrosis or chronic obstructive pulmonary disease (COPD).

11. The method of claim 8, wherein the bacterial infection of the gastrointestinal tract causes Crohn's disease.

12. The method of claim 8, wherein the condition is caused by Gram positive and/or Gram negative bacteria, wherein the Gram positive and/or Gram negative bacteria are selected from the group consisting of Staphylococcus such as Staphylococcus aureus and methicillin-resistant Staphylococcus aureus (MRSA), Mycobacterium such as Mycobacterium tuberculosis such as multidrug-resistant (MDR) and extensively drug-resistant (XDR) strains, Pseudomonas such as Pseudomonas aeruginosa and multidrug-resistant Pseudomonas aeruginosa (MDRPA), Enterococcus such as vancomycin-resistant Enterococcus (VRE), Haemophilus such as Haemophilus influenzae, E. coli such as extended-spectrum beta-lactamase (ESBL) producing E. coli, Klebsiella such as Klebsiella pneumonia, β-hemolytic Streptococcus such as group A Streptococcus such as Streptococcus pyogenes, and Acinetobacter such as Acinetobacter baumannii.


SEQUENCE LISTING (2 sequences)

SEQ ID NO: 1 - protein, 19 aa, synthetic construct
  REGION 1..19: note = Peptide 19-4LF
GKKYRRFRWKFKGKLFLFG

SEQ ID NO: 2 - protein, 20 aa, synthetic construct
  REGION 1..20: note = Peptide 19-2.5
GCKKYRRFRWKFKGKFWFWG